(19) United States
(12) Patent Application Publication
(10) Pub. No.: US 2011/0251239 A1
(43) Pub. Date: Oct. 13, 2011

(54) COMBINATION THERAPY FOR THE TREATMENT OF DEMENTIA

(75) Inventors: Margaret Lynn MOLINE, White Plains, NY (US); James FERRY, Hampton, NJ (US); Timothy HSU, Jackson Heights, NY (US); Lynn Darden KRAMER, Westport, CT (US); Andrew SATLIN, Short Hills, NJ (US)

(73) Assignee: EISAI INC., Woodcliff Lake, NJ (US)

(21) Appl. No.: 13/081,810

(22) Filed: Apr. 7, 2011

Related U.S. Application Data
(60) Provisional application No. 61/321,700, filed on Apr. 7, 2010.

(51) Int. Cl. A61K 31/445; A61P 25/28

(52) U.S. Cl. 514/319

(57) ABSTRACT

Provided herein is a combination therapy useful for the treatment of Alzheimer's disease and Alzheimer's disease related dementia. The combination comprises memantine in an immediate or sustained release form and donepezil in a sustained release form.

### RELATED APPLICATIONS

This application claims priority to U.S. Provisional Application No. 61/321,700, filed on Apr. 7, 2010, titled “Combination Therapy for the Treatment of Dementia”. The entire contents of the foregoing application are incorporated herein by reference.

### BACKGROUND

Alzheimer's disease is a progressive neurodegenerative disorder that leads to the death of brain cells and a progressive clinical dementia. The neuropathology is characterized by the presence of amyloid plaques, neurofibrillary tangles, synaptic loss and selective neuronal cell death. The plaques are a result of abnormal levels of extracellular amyloid beta peptide while the tangles are associated with the presence of intracellular hyperphosphorylated tau protein. Symptoms first manifest clinically with a decline in memory followed by deterioration in other cognitive functions and by abnormal behavior. Age is the single most prominent risk factor, with the incidence doubling every five years from approximately 1.6% at the age of 65-74, with the rate increasing to 19% in the 75-84 group and to 42% in the greater than 84 group. Prevalence studies estimate that in 2000 the number of persons with Alzheimer's disease in the US alone was 4.5 million, with numbers expected to increase almost 3-fold in the next 50 years due to the rapid growth of the oldest age groups. The increasing number of dementia patients in the developed world will place an enormous burden on society and the health care systems.

Donepezil is a centrally acting reversible acetylcholinesterase inhibitor (AChEI). See e.g., U.S. Pat. Nos. 4,895,841, 5,985,864, 6,140,321, 6,245,911, 6,372,760, and 6,458,807. Aricept® (donepezil hydrochloride) is currently indicated for mild to moderate and severe dementia due to Alzheimer's. See, http://www.aricept.com/images/AriceptComboFullPINovember02006.pdf

Memantine acts on the glutamatergic system by blocking NMDA glutamate receptors. See e.g., U.S. Pat. Nos. 3,391,142, 4,122,193; 4,273,774, 5,061,703, 5,891,885, 5,919,826, and 6,187,338.

AChEIs, such as donepezil, galantamine and rivastigmine have been used in combination with memantine. See, e.g., Yao et al. J. Clin. Pharmacol., 2005, 45, pp. 519-528, Shua-Haim et al., Clin. Drug Invest., 2008, 28, 6, pp. 361-374, and Periclou, A. P. et al., The Annals of Pharmacotherapy, 2004, 38, pp. 1389-1394. However, the clinical trial that was submitted for the global approvals of memantine was conducted with donepezil and memantine or memantine placebo.

Despite recent advances in the understanding and treatment of Alzheimer's disease, there is an unmet need to provide a simple and effective therapy. For example, there is a need for methods and/or compositions for treating Alzheimer's disease that lower the pill burden on the patient, lower the burden on the caregiver, and allow the patient to enjoy a better quality of life without the need for institutional care and/or hospitalization.

### SUMMARY OF THE INVENTION

There remains a need for new treatments and therapies for Alzheimer's disease, and Alzheimer's disease-related disorders, such as dementia. There is also a need for compounds useful in the treatment or prevention or amelioration of one or more symptoms of Alzheimer's disease, and Alzheimer's disease-related dementia.

Thus, provided herein is a combination therapy, comprising an effective amount of memantine, and an effective amount of donepezil, wherein the donepezil is in a sustained release formulation. Surprisingly and unexpectedly, the combination therapy provided herein shows a synergistic effect that increases the exposure of donepezil in a patient. The synergistic effect is accompanied by an increase in the plasma concentration of donepezil, when donepezil is administered in combination with memantine. The combination therapy also surprisingly provides an improvement in Severe Impairment Battery (SIB) of patients being treated or moderately-severe to severe Alzheimer's disease with an acceptable adverse event profile. In particular, a 21-24 mg dose of donepezil in combination with memantine surprisingly increases the exposure of donepezil and provides an unexpected improvement in the SIB of patients being treated for moderately-severe to severe Alzheimer's disease. Additionally, the 21-24 mg dose of donepezil in combination with memantine is surprisingly well tolerated by patients being treated for moderately-severe to severe Alzheimer's disease. Moreover, patients who were administered a combination of donepezil and memantine showed a surprising and unexpected 21% lower apparent clearance of donepezil compared to patients who were administered donepezil alone. An advantage of this lower apparent clearance is the increase in the plasma concentration (“exposure”) of donepezil of approximately 20%. This is equivalent to increasing the dose of donepezil by 20%.

In one aspect, provided herein is a method of treating moderate to severe Alzheimer's disease in a patient in need thereof. The method comprises administering to the patient a daily dose of (i) 21-24 mg of donepezil, or any pharmaceutically acceptable salt thereof, and (ii) 20 mg of memantine, or any pharmaceutically acceptable salt thereof.

In some embodiments, the method comprises administering 23 mg of donepezil. In various embodiments, the donepezil is formulated as an immediate release formulation. In certain embodiments, the donepezil is formulated as a sustained release formulation.

In certain embodiments of the method, the donepezil is formulated as an immediate release formulation comprising 23 mg of donepezil. In some embodiments, the donepezil is formulated as a sustained release formulation comprising 23 mg of donepezil.

In various embodiments of the method, the patient has a Mini-Mental State Exam (MMSE) score of 0-20. In some embodiments, the patient has a MMSE score of 0-16. In another embodiment, the patient has a MMSE score of 0-10.

In some embodiments of the method, the memantine is formulated as an immediate release formulation comprising 10 mg of memantine, and administered twice daily. In some embodiments, the memantine is formulated as a sustained release formulation comprising 10 mg of memantine, and administered twice daily. In certain embodiments, the memantine is formulated as a sustained release formulation comprising 20 mg of memantine.

In certain embodiments of the method, the donepezil and memantine are administered simultaneously, separately or sequentially. In some embodiments, the daily dose of memantine comprises two unit doses of 10 mg each. In various embodiments, the donepezil and memantine is formulated as a single formulation. In some embodiments, the donepezil is donepezil hydrochloride. In certain embodiments, the memantine is memantine hydrochloride. In various embodiments of the method, the patient has severe Alzheimer's disease. In some embodiments, the treatment comprises slowing the symptomatic progression of moderate to severe Alzheimer's disease.

In another aspect, provided herein is a method of treating moderate to severe Alzheimer's disease in a patient with a MMSE score of 0-16, comprising administering to the patient (i) a sustained release formulation comprising 23 mg of donepezil, or any pharmaceutically acceptable salts thereof, once daily and (ii) an immediate release formulation comprising 10 mg of memantine, or any pharmaceutically acceptable salts thereof, twice daily.

In yet another aspect, provided herein is a method of treating moderate to severe Alzheimer's disease in a patient with an MMSE score of 0-16, comprising administering to the patient a sustained release formulation comprising (i) 23 mg of donepezil, or any pharmaceutically acceptable salts thereof, and (ii) 20 mg of memantine, or any pharmaceutically acceptable salts thereof, wherein the formulation is a single formulation or unit dose.

In another aspect, provided herein is a pharmaceutical formulation comprising (i) 21-24 mg (e.g., 23 mg) of donepezil, or any pharmaceutically acceptable salts thereof, (ii) 20 mg of memantine, or any pharmaceutically acceptable salts thereof, and (iii) a pharmaceutically acceptable carrier or excipient, wherein the formulation is a single formulation or unit dose. In certain embodiments, the pharmaceutical formulation comprises one or more pharmaceutically acceptable carriers or excipients.

In yet another aspect, provided herein is a pharmaceutical formulation comprising (i) 10 mg of donepezil, or any pharmaceutically acceptable salts thereof, (ii) 20 mg of memantine, or any pharmaceutically acceptable salts thereof, and (iii) a pharmaceutically acceptable carrier or excipient, wherein the formulation is a single formulation or unit dose. In some embodiments, the formulation is an immediate release formulation. In certain embodiments, the donepezil is formulated as a sustained release formulation and the memantine is formulated as an immediate release formulation. In various embodiments, both active ingredients are formulated as a sustained release formulation.

In certain embodiments of the methods and formulations provided herein, the donepezil is donepezil hydrochloride. In other embodiments, the memantine is memantine hydrochloride.

In another aspect, provided herein is an immediate release formulation comprising (i) 10 mg of donepezil hydrochloride, (ii) 20 mg of memantine hydrochloride, and (iii) a pharmaceutically acceptable carrier or excipient, wherein the formulation is a single formulation or unit dose. In certain embodiments, the donepezil is in a sustained release formulation.

In yet another aspect, provided herein is a sustained release formulation comprising (i) 23 mg of donepezil hydrochloride, (ii) 28 mg of memantine hydrochloride, and (iii) a pharmaceutically acceptable carriers or excipients, wherein the formulation is a single formulation or unit dose.

In some embodiments, the donepezil and memantine are formulated as a paste, jelly, or suspension. For example, the drugs are dissolved, entrapped or suspended in the form of drug particles, microencapsulated particles, or drug-polymer particles in a gelatinous solution or semi-solid. In certain embodiments, both the donepezil and memantine are thoroughly mixed and suspended in an appropriate medium to form a paste or a gel. In various embodiments, the paste or jelly is formulated with suitable binders or excipients known in the art for topical administration. Typically, topical administration includes applying the formulation over a portion of skin, i.e., transdermally. One skilled in the art appreciates that any suitable portion of the skin can be selected for topical administration, for example forehead.

The foregoing brief summary broadly describes the features and technical advantages of certain embodiments of the present invention. Further technical advantages will be described in the detailed description of the invention that follows. The figures and examples provided herein are intended to help illustrate the invention or assist with developing an understanding of the invention, and are not intended to be definitions of the invention's scope.

### BRIEF DESCRIPTION OF THE DRAWINGS

FIG. 1 shows improvements in the Severe Impairment Battery (SIB) between patients receiving a daily dose of 10 mg of donepezil (immediate release) and patients receiving a daily dose of 23 mg of donepezil (sustained release) during a clinical trial, discussed herein.

FIG. 2 shows improvements in severe impairment battery (SIB) between patients receiving a daily dose of 10 mg of donepezil (immediate release) and patients receiving a daily dose of 23 mg of donepezil (sustained release) during a clinical trial discussed herein.

FIG. 3 shows the dose normalized donepezil concentration between patients receiving a once daily dose of 23 mg of donepezil (sustained release) along with a twice daily dose of 10 mg each of memantine (immediate release) vs. patients receiving only the once daily dose of 23 mg of donepezil (sustained release).

FIG. 4 shows the dose normalized donepezil concentration between patients receiving a once daily dose of 10 mg of donepezil (immediate release) along with a twice daily dose of 10 mg each of memantine (immediate release) vs. patients receiving only the once daily dose of 10 mg of donepezil (immediate release).

FIG. 5 shows a box and whisker plot of the inter-individual variability on apparent clearance (IIVCL) (as depicted by the Y-axis) for both immediate release and sustained release formulations versus memantine co-administration status. The central line in the box represents the median value in the distribution of individual values, the lower edge of the box represents the 25^{th} percentile, the upper edge of the box represents the upper 75^{th} percentile and the lower and upper “whiskers” or lines radiating from the box represent the lower 2.5^{th} percentile and the upper 97.5^{th} percentile, respectively. The stars represent outliers in each distribution as determined by the statistical program used to generate the figure.

### DETAILED DESCRIPTION OF THE INVENTION

Prior to setting forth the invention in detail, it may be helpful to provide definitions of certain terms to be used herein. Unless defined otherwise, all technical and scientific terms used herein have the same meaning as is commonly understood by one of skill in the art to which this invention belongs.

Provided herein is an invention for a combination of therapeutic agents and administration of the combination of agents to treat Alzheimer's disease, as well as Alzheimer's disease-related dementia. As used herein, a “combination of therapeutic agents” and similar terms refer to a combination of two types of therapeutic agents: (1) memantine and/or pharmacologically active metabolites, salts, solvates and racemates of memantine and (2) donepezil, and/or pharmacologically active metabolites, salts, solvates and racemates of donepezil. Pharmacologically active metabolites comprise those that are inactive but converted into pharmacologically active forms in the body after administration.

Donepezil ([(R,S)-1-benzyl-4-[(5,6-dimethoxy-1-indanon)-2-yl]-methylpiperidine hydrochloride], also known as Aricept®) is a reversible, noncompetitive, piperidine-type acetylcholinesterase inhibitor. Studies have shown that daily administration of donepezil (5 and 10 mg/day) can lead to significantly improved cognition and global clinical function compared with placebo in short and long-term trials. Donepezil is described, for example, in U.S. Pat. Nos. 6,372,760; 6,245,911; 6,140,321; 5,985,864; and 4,895,841, all of which are incorporated herein by reference in their entireties.

Memantine (1-amino-3,5-dimethyl adamantane) is described, for example, in U.S. Pat. Nos. 4,122,193; 4,273,774; 5,061,703, all of which are incorporated herein by reference in their entireties. Memantine is an Alzheimer's disease medication acting on the glutamatergic system by blocking NMDA glutamate receptors. Memantine is advantageous because it lacks the side effects of other NMDA receptor antagonists at similar therapeutic doses.

Administration of the combination of therapeutic agents comprises administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agents as compared to the other agent in the combination. Therefore, to permit appropriate dosing, packaged pharmaceutical products may contain one or more dosage forms that contain the combination of agents, and one or more dosage forms that contain one of the combinations of agents, but not the other agent(s) of the combination.

Therapeutic agents may contain one or more asymmetric elements such as stereogenic centers or stereogenic axes, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with carbon-carbon double bonds may occur in Z- and E-forms; all isomeric forms of the compounds are included in the present invention. In these situations, the single enantiomers (optically active forms) can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

Unless otherwise specified, or clearly indicated by the text, reference to compounds useful in the combination therapy of the invention includes both the free base of the compounds, and all pharmaceutically acceptable salts of the compounds. A preferred memantine salt is the hydrochloride salt. A preferred donepezil salt is the hydrochloride salt. The terms “memantine or pharmaceutically acceptable salts thereof,” “donepezil or pharmaceutically acceptable salts thereof” and the like, indicate the pharmaceutically acceptable salts of memantine and donepezil, respectively.

The term “pharmaceutically acceptable salts” comprises derivatives of the disclosed compounds, wherein the parent compound is modified by making non-toxic acid or base addition salts thereof, and further refers to pharmaceutically acceptable solvates, comprising hydrates, of such compounds and such salts. Examples of pharmaceutically acceptable salts comprise, but are not limited to, mineral or organic acid addition salts of basic residues such as amines; alkali or organic addition salts of acidic residues such as carboxylic acids; and the like, and combinations comprising one or more of the foregoing salts. Pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, and nitric; other acceptable inorganic salts include metal salts such as sodium salt, potassium salt, and cesium salt; and alkaline earth metal salts, such as calcium salt and magnesium salt; and combinations comprising one or more of the foregoing salts.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC(CH₂)ₙCOOH where n is 0-4; organic amine salts such as triethylamine salt, pyridine salt, picoline salt, ethanolamine salt, triethanolamine salt, dicyclohexylamine salt, N,N′-dibenzylethylenediamine salt; and amino acid salts such as arginate, asparginate, and glutamate, and combinations comprising one or more of the foregoing salts.

An “effective amount” of a combination of therapeutic agents (e.g., donepezil and memantine) is an amount sufficient to provide an observable therapeutic benefit compared tobaseline clinically observable signs and symptoms of Alzheimer's disease, and Alzheimer's disease-related dementia treated with the combination.

“Immediate-release” is meant to include a conventional release, in which release of the drug starts immediately after administration. As used herein, the term “immediate release” includes dosage forms that allow the drug to dissolve in the gastrointestinal contents, with no intention of delaying or prolonging the dissolution or absorption of the drug. The objective is for the drug to be released rapidly after administration, for example for it to be possible to release at least 80% of the anti-dementia drug within approximately 30 minutes after commencement of dissolution in a dissolution test.

“Sustained-release” or “extended-release” includes dosage forms whose drug-release characteristics of time course and/or location are chosen to accomplish therapeutic or convenience objectives not offered by conventional dosage forms such as a solution or an immediate release dosage form.

The term “steady-state” means that a plasma level for a given active agent or combination of active agents, has been achieved and which is maintained with subsequent doses of the active agent(s) at a level which is at or above the minimum effective therapeutic level and is below the minimum toxic plasma level for a given active agent(s).

The term “single formulation” as used herein refers to a single carrier or vehicle formulated to deliver effective amounts of both therapeutic agents to a patient. The single vehicle is designed to deliver an effective amount of each of the agents, donepezil and memantine along with any pharmaceutically acceptable carriers or excipients. In some embodiments, the vehicle is a tablet, capsule, pill, or a patch.

The term “unit dose” is used herein to mean simultaneous administration of both agents together, in one dosage form, to the patient being treated. In some embodiments, the unit dose is a single formulation. In certain embodiments, the unit dose includes one or more vehicles such that each vehicle includes an effective amount of at least one of the agents (donepezil or memantine) along with pharmaceutically acceptable carriers and excipients. In some embodiments, the unit dose is one or more tablets, capsules, pills, or patches administered to the patient at the same time.

The term “dose range” as used herein refers to an upper and a lower limit of an acceptable variation of the amount of agent specified. Typically, a dose of the agent in any amount within the specified range can be administered to patients undergoing treatment.

The term “treat” is used herein to mean to relieve, reduce or alleviate at least one symptom of a disease in a subject. For example, in relation to dementia, the term “treat” may mean to relieve or alleviate cognitive impairment (such as impairment of memory and/or orientation) or impairment of global functioning (overall functioning, including activities of daily living) and/or slow down or reverse the progressive deterioration in global or cognitive impairment. Within the meaning of the present invention, the term “treat” also denotes, to arrest, delay the onset (i.e., the period prior to clinical manifestation of a disease or symptom of a disease) and/or reduce the risk of developing or worsening a symptom of a disease. The term “protect” is used herein to mean prevent delay or treat, or all, as appropriate, development or continuance or aggravation of symptoms of the disease in a subject. Within the meaning of the present invention, the dementia is associated with a CNS disorder, including without limitation neurodegenerative diseases such as Alzheimer's disease, Lewy body dementia, Down's syndrome and cerebrovascular dementia. In a particular embodiment, the dementia is associated with Alzheimer's disease.

The term “subject” is intended to include animals, which are capable of suffering from or afflicted with dementia associated with a CNS disorder, including without limitation neurodegenerative diseases such as Alzheimer's disease, Down's syndrome and cerebrovascular dementia, or any disorder involving, directly or indirectly, Alzheimer's disease. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from Alzheimer's disease or Alzheimer's disease-associated dementia, or Lewy body dementia.

The term “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term “synergistic effect” as used herein, refers to action of two agents such as, for example, donepezil and memantine, producing an effect, for example, slowing the symptomatic progression of Alzheimer's disease or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S, and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

Methods of Treatment

Provided herein is a combination therapy useful for the treatment of moderately-severe to severe Alzheimer's disease, as well as symptoms associated with moderately-severe to severe Alzheimer's disease. As discussed below, the combination provided herein has a number of advantages.

One advantage of the combination disclosed herein is the unexpected synergistic effect of memantine on the increased exposure of donepezil. Donepezil belongs to a class of acetylcholinesterase inhibitors (AChEIs). The metabolic pathway of donepezil involves degradation by cytochrome P450. Donepezil is metabolized primarily by CYP3A4 and, to a lesser extent, by CYP2D6. See, e.g., Yao et al. J. Clin. Pharmacol., 2005, 45, pp. 519-528.

Memantine belongs to a class of N-methyl-D-aspartate (NMDA) antagonists. The metabolic pathway of memantine is not the same as that of AChEIs, and memantine produces minimal inhibition of CYP enzymes. Therefore, administering memantine in combination with other AChEIs such as galantamine or rivastigmine did not lead to any alteration of their metabolism. See, e.g., Shua-Haim et al., Clin. Drug Invest., 2008, 28, 6, pp. 361-374.

Previous studies using 5 mg and 10 mg of donepezil in combination with memantine indicated that the two drugs could be safely administered to subjects with no significant effects on the pharmacokinetic profile of either drug. See, e.g., Periclou, A. P. et al., The Annals of Pharmacotherapy, 2004, 38, pp. 1389-1394. One of skill in the art appreciates that the results obtained in previous studies cannot be extrapolated to predict the effect of a higher dose of donepezil on pharmacokinetic profile.

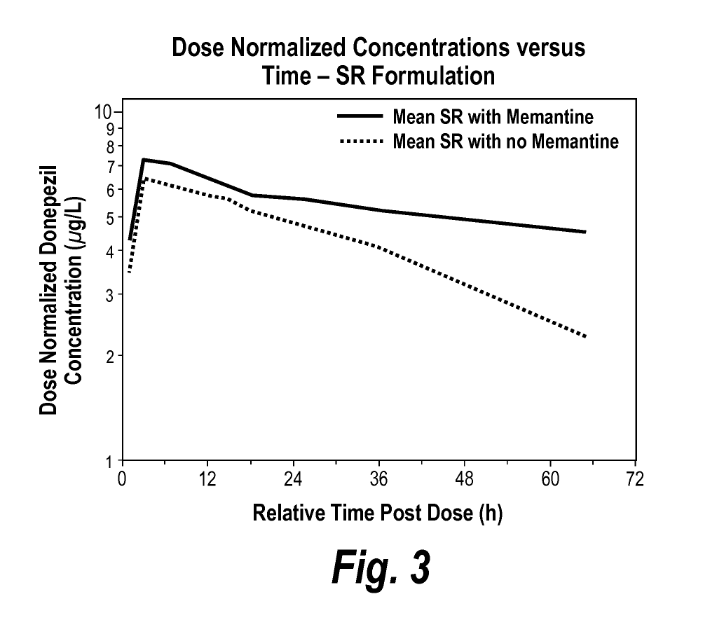

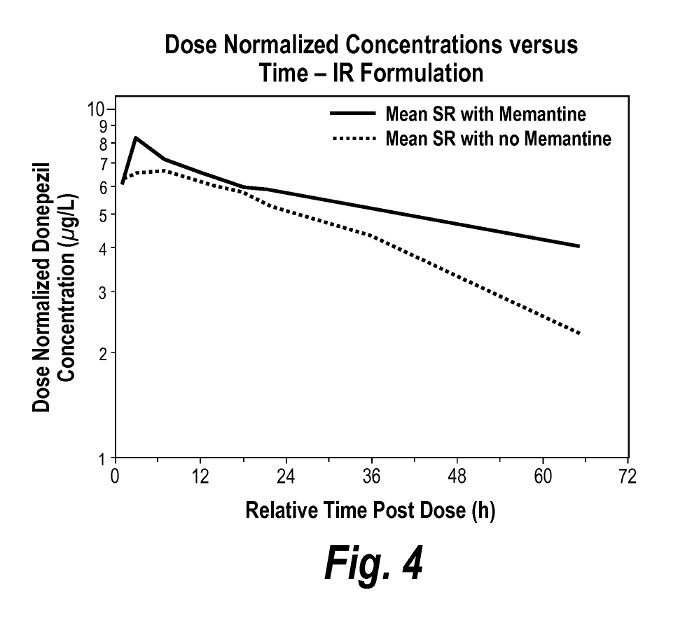

Surprisingly and unexpectedly, it was found that when donepezil and memantine were administered to patients as a combination treatment for moderately-severe to severe Alzheimer's disease, the rate of metabolism of donepezil was slower in these patients than when donepezil was administered alone. In particular, the rate of metabolism of donepezil administered once a day as either a 23 mg sustained release formulation or a 10 mg immediate release formulation to patients taking 10 mg of memantine, twice a day, was slower than the same formulation of donepezil administered to patients not taking memantine. See, e.g., FIGS. 3 and 4. These unexpected results show that the combination treatment of the invention leads to an increase in the plasma concentration or exposure of donepezil. This exposure is equivalent to approximately 20% increase in the donepezil dose.

Surprisingly and unexpectedly, it was found that when donepezil and memantine were administered to patients as a combination treatment for moderately-severe to severe Alzheimer's disease, a 21% lower apparent clearance of donepezil was obtained compared to patients who were administered donepezil alone. An advantage of this lower apparent clearance apart from the prolongation of the half-life of donepezil, is the increase in the plasma concentration or exposure of donepezil. This exposure is equivalent to approximately 20% increase in the donepezil dose.

Furthermore, it should be noted that previous studies conducted for treatment of Alzheimer's disease using a combination of donepezil and memantine, compared the effect of the combination treatment to that of a placebo. See e.g., Periclou, A. P. et al., The Annals of Pharmacotherapy, 2004, 38, pp. 1389-1394. In contrast, the studies conducted for the combination treatment provided herein compare the effect of a combination treatment to an already approved drug or active agent, and not a placebo, indicating that the increase in blood levels is sustained even in the long term. These results indicate that administration of donepezil once a day as a 23 mg sustained release formulation to patients taking 10 mg of memantine, twice a day, is even more effective than the presently prevalent therapy of 10 mg donepezil combined with 20 mg memantine (10 mg, twice daily). Surprisingly, and unexpectedly, this combination therapy comprising a high dose of donepezil was well tolerated by patients participating in the trial discussed above. Prior to the clinical trial, it was unknown if a substantial group of patients undergoing treatment for moderate to severe Alzheimer's disease would tolerate a higher dose of donepezil.

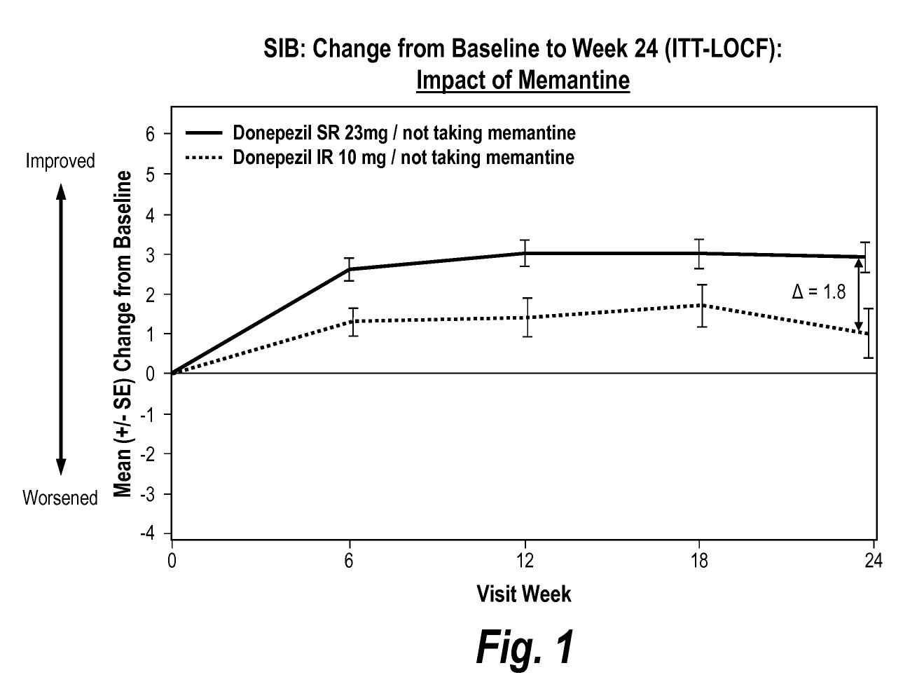

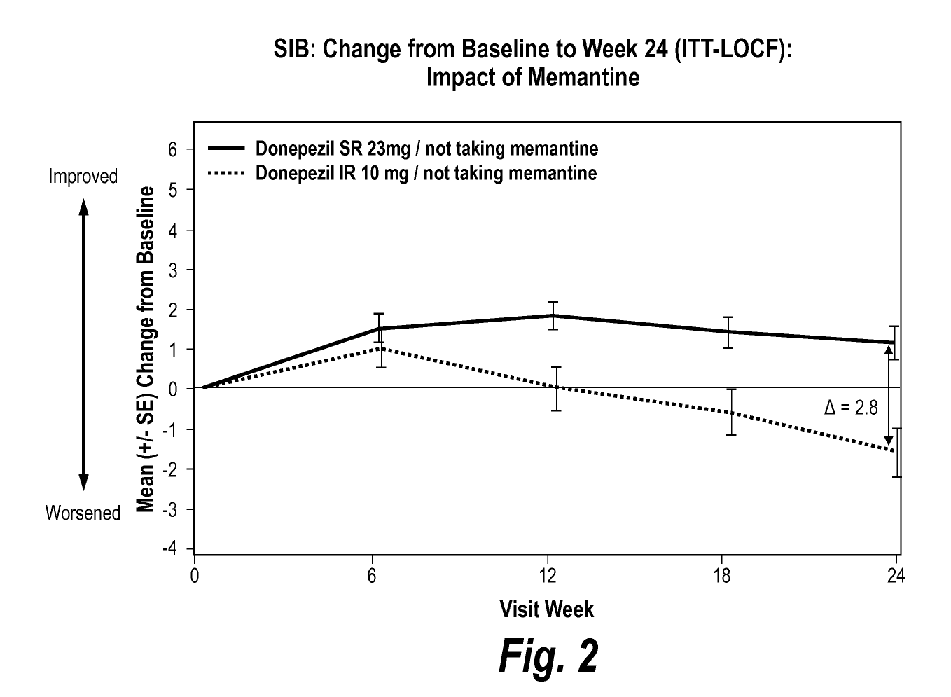

Other unexpected benefits are shown in FIGS. 1 and 2. FIG. 1 shows improvements in the Severe Impairment Battery (SIB) between patients receiving a daily dose of 10 mg of donepezil (immediate release) and patients receiving a daily dose of 23 mg of donepezil (sustained release) during a clinical trial. In this trial, 1467 patients were randomized with approximately 2:1, 23 mg:10 mg of donepezil administration. Co-primary endpoints were the Severe Impairment Battery (SIB, cognitive endpoint) and Clinician's Interview-Based Impression of Change plus caregiver input (CIBIC+, endpoint for global function).

FIG. 2 compares improvements in Severe Impairment Battery (SIB) between patients receiving a daily dose of 10 mg of donepezil (immediate release) and patients receiving a daily dose of 23 mg of donepezil (sustained release). In this trial, patients were concurrently allowed to take memantine (over 90% of patients took 10 mg, twice daily). In this trial patients had been on memantine for at least 3 months before the trial commenced. Compared with FIG. 1, a greater improvement in SIB was observed for patients taking 23 mg of sustained release donepezil in combination with memantine as compared to those patients not taking memantine (A of 2.8 vs. 1.8). See FIG. 3. The magnitude of the improvement in these patients is particularly surprising, and demonstrates that treatment of moderate to severe Alzheimer's disease can be improved by increasing the dose of donepezil administered in combination with memantine. Therefore, an unexpected synergistic effect exists when a higher dose of donepezil is used in the combination with memantine to treat moderate to severe Alzheimer's disease.

As such, the high dose combination therapy described herein provides an additional therapeutic benefit for patients who are already receiving the maximum dose of donepezil and memantine as currently approved. Such patients typically have no alternative therapy available that can provide equal or better treatment that is well tolerated and has an acceptable adverse event profile.

In some embodiments, provided herein is a single pharmaceutical formulation containing a combination of donepezil and memantine. An advantage provided herein is the synergistic effect that results in the treatment of Alzheimer's disease, including Alzheimer's disease-associated dementia, compared to treatment with a single dose of either drug. When the drugs are provided in a single unit dose or single formulation, the “pill burden” on a patient suffering from Alzheimer's disease is substantially reduced. Moreover, the burden on caregivers of patients suffering from Alzheimer's disease is also reduced. The burden placed on caregivers of patients suffering from Alzheimer's disease is substantial, because the patients are forced to rely upon the caregiver to administer the appropriate number and dosage of pills, as well as monitor the appropriate time of administration.

As specified above, in one aspect, the provided herein is a drug combination useful for treating, preventing, arresting, delaying the onset of and/or reducing the risk of developing, or reversing at least one symptom of dementia associated with a central nervous system disorder, especially Alzheimer's disease, Lewy body dementia, dementia associated with cerebrovascular disease, or Down's Syndrome, in a mammal comprising administering to said mammal a combination therapy, comprising an effective amount of memantine, and an effective amount of donepezil. Preferably, the donepezil is administered as a sustained release formulation. The donepezil and the memantine are administered at therapeutically effective dosages which, when combined, provide a beneficial effect.

The combination provided herein can also be used to treat moderate to severe Alzheimer's (or dementia associated with moderate to severe Alzheimer's) in a patient in need thereof. As such, the combination has the advantage of prolonging the onset of the most severe stage of the disease. This results in an improved quality of life for the patient and caregiver, as well as delaying the need to provide the patient with institutional care and/or hospitalization.

The term “Alzheimer's disease” refers to a progressive disease of the human central nervous system. It is manifested by dementia typically in the elderly, by disorientation, loss of memory, difficulty with language, calculation, or visual-spatial skills, and by psychiatric manifestations. It is associated with degenerating neurons in several regions of the brain. The term “dementia” as used herein includes, but is not restricted to, Alzheimer's dementia with or without psychotic symptoms.

In a particular embodiment, the therapeutic combination provided herein is effective for the treatment of moderate to severe Alzheimer's disease in a subject. These phases of Alzheimer's include “moderately severe cognitive decline,” also referred to as “moderate or mid-stage Alzheimer's disease;” “severe cognitive decline,” also referred to as “moderately severe or mid-stage Alzheimer's disease;” and “very severe cognitive decline,” also referred to as “severe or late-stage Alzheimer's disease.” Moderately severe cognitive decline is characterized by major gaps in memory and deficits in cognitive function emerge. At this stage, some assistance with day-to-day activities becomes essential. In severe cognitive decline, memory difficulties continue to worsen, significant personality changes may emerge and affected individuals need extensive help with customary daily activities. Finally, late stage Alzheimer's disease or very severe cognitive decline is the final stage of the disease when individuals lose the ability to respond to their environment, the ability to speak and, ultimately, the ability to control movement. (See, http://www.alz.org). In certain embodiments the phase of moderately-severe to severe Alzheimer's disease can be referred to as “advanced Alzheimer's disease”.

In another embodiment, the patient to be treated by the combination therapy of the invention has an MMSE score between 0 and 20. “MMSE” refers to the Mini-Mental State Examination used in the cognitive assessment community. In general, a patient with an MMSE score of 27-30 is considered to have no or mild cognitive impairment, a patient with an MMSE score of 21-26 is considered to have mild dementia, and a patient with an MMSE score of 0-12 is considered to have severe cognitive impairment. In certain embodiments, a patient with an MMSE score of 0-16 is considered to have advanced (moderately severe to severe) Alzheimer's disease.

In some embodiments, the subject to be treated (e.g., patient) was determined to be non-responsive or resistant to one or more Alzheimer's disease therapies, e.g., memantine. In other embodiments, the individual to be treated was responsive to memantine therapy, but the therapy was improved with the administration of donepezil. For example, the patient is administered memantine (e.g., up to 50 mg per day, preferably up to 40 mg per day, more preferably up to 30 mg per day) for some period of time, e.g., more than one day, more than two days, more than three days, more than one week, more than one month, etc. After that time, donepezil (e.g., up to 50 mg per day, preferably up to 40 mg per day, more preferably up to 30 mg per day in a sustained release form) could be administered to that patient in combination with memantine. In certain embodiments, 21-24 mg, e.g., approximately 23 mg, per day of donepezil can be administered. In various embodiments, the approximately 23 mg of donepezil is in a sustained release formulation. The two drugs could be administered at the same time in a single dosage formulation, at the same time in separate dosage formulations, or at separate times in separate dosage formulations, but within 24 hours of one another. In a particular embodiment, the patient is receiving up to 20 mg or 28 mg of memantine, wherein the memantine is in an immediate release or sustained release formulation, and approximately 10 mg or 23 mg of donepezil, wherein the donepezil is in a sustained release formulation.

One skilled in the art appreciates that, the effective dose of the active drug may be lower that the actual amount administered. As such, provided herein are doses necessary to achieve a therapeutic dose. For example, in a non-limiting embodiment, approximately 23 mg of donepezil can be administered to achieve approximately 19 mg of donepezil available for efficacy.

In various embodiments, provided herein are methods of treating Alzheimer's-related dementia by administering an effective amount of memantine and donepezil, to an individual having Alzheimer's, e.g., moderate to severe Alzheimer's disease. The amount of the combination of agents (i.e., memantine and donepezil) is effective to treat the Alzheimer's-related dementia (e.g., moderate to severe Alzheimer's-related dementia). In one embodiment, the combination of agents has a synergistic effect. In one embodiment, even though one or more of the agents administered alone at a particular dosage may be effective, when administered in combination, at the same dosage of each agent, the treatment is more effective. For example, when a patient is administered a combination of memantine and donepezil, treatment is more effective with both agents than with administration of either agent alone. In another example, treatment with a combination of memantine (in an immediate release or sustained release formulation) and donepezil in a sustained release formulation is more effective than administration of a combination of memantine in an immediate release formulation and donepezil in an immediate release formulation. In another embodiment, combining memantine with a higher dose of donepezil (e.g., 20 mg or more per day, e.g., 20-26 mg per day, preferably 21-24 mg, more preferably 23 mg per day) in a sustained release form shows a greater effect on dementia than a higher dose of donepezil in a sustained release form without concurrent memantine administration.

Particularly preferred methods include delaying the progression of symptoms of a dementia associated with moderate to severe Alzheimer's disease, or treating such dementia comprising administering to a subject an effective amount of memantine (in an immediate release or sustained release formulation), and an effective amount of donepezil, in a sustained release formulation, and administered at a dose of 20 mg or more (e.g., 20-26 mg, preferably 21-24 mg, more preferably 23 mg) per day.

Dosages

The optimal dose of the combination of agents for treatment of Alzheimer's-related dementia can be determined empirically for each individual using known methods and will depend upon a variety of factors, including the activity of the agents; the age, body weight, general health, gender and diet of the individual; the time and route of administration; and other medications the individual is taking. Optimal dosages may be established using routine testing and procedures that are well known in the art.

Typically, the daily dose of memantine is 20 mg per day. This dose is achieved by upward titration 5 mg per week over the first 3 weeks as follows: treatment should be started with 5 mg daily (half a tablet in the morning) during the 1st week. In the 2nd week 10 mg per day (half a tablet twice a day) and in the 3rd week 15 mg per day is recommended (one tablet in the morning and half a tablet in the afternoon). From the 4th week on, treatment can be continued with the recommended maintenance dose of 20 mg per day (one tablet twice a day). The recommended starting dose for donepezil for patients with Alzheimer's disease is 5 mg once a day. Typically, this dose is increased to 10 mg once daily after the patient has been taking the 5 mg dose for at least four to six weeks. Current donepezil therapy uses a single maximum dose of 10 mg.

For the combination therapy of the instant invention, the daily dose of memantine is in the range of 5 to 50 mg. In some embodiments, the daily dose of memantine is up to 50 mg per day. In certain embodiments, the daily dose of memantine is up to 40 mg per day. In various embodiments, the daily dose of memantine is up to 30 mg per day. In certain embodiments, the daily dose of memantine is 20 mg or 28 mg per day. In one embodiment, the daily dose is in the range of 5-20 mg. In still another embodiment, the daily dose is approximately 10 mg or approximately 20 mg. In various embodiments, the daily dose of memantine is in the range of 24 to 32 mg. In certain embodiments, the daily dose of memantine is 28 mg per day. In some embodiments, the daily dose of memantine is 28 mg per day, in a sustained release formulation. In another embodiment, the memantine is contained in an immediate release or sustained release formulation.

For the combination therapy of the instant invention, the daily dose of donepezil is in the range of 5 to 50 mg. In some embodiments, the daily dose of donepezil can be in the range of 10 to 50 mg. In certain embodiments, the daily dose of donepezil can be in the range of 10-20 mg, 20 to 30 mg, or 30-40 mg. In some embodiments, the daily dose of donepezil can be in the range of 15-40 mg, or 40-50 mg. In various embodiments, the daily dose of donepezil can be in the range of and 20-35 mg. In one embodiment, the daily dose is in the range of 20-26 mg. In still another embodiment, the daily dose is approximately 23 mg. In a particularly preferred embodiment, the donepezil is in a sustained release formulation.

The time of administration can be chosen such that both the drugs are administered simultaneously, separately or sequentially, either in the morning or at night. Alternatively, one drug can be administered in the morning and the other at night. In certain embodiments, both the drugs can be administered as a single tablet, capsule, pill, patch or jelly formulation, once daily, either in the morning or at night.

Smaller doses of memantine can be administered as appropriate (e.g., to individuals with other medical conditions). The daily dose will vary from individual to individual and from time to time for a given individual (e.g., as daily dose is adjusted with the individual's changing mental states or general health).

The amount of combination of agents that may be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

Frequency of dosage may vary depending on the compound used and the particular condition to be treated or prevented. In general, for treatment of most Alzheimer's-related disorders, a dosage regimen of 4 times daily or less is preferred. For the treatment of Alzheimer's-related disorders, including moderate to severe Alzheimer's-related disorders, a dosage regimen of 1 or 2 times daily is particularly preferred. A single dose per day is most preferred. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Pharmaceutical Formulations and Routes of Administration

Provided herein are pharmaceutical formulations comprising a combination of agents for the treatment of Alzheimer's-related disorders, e.g., Alzheimer's-related dementia. The pharmaceutical formulations may additionally comprise a carrier or excipient, stabilizer, flavoring agent, and/or coloring agent.

Provided herein are pharmaceutical formulations comprising combination of agents which can be, for example, a combination of two types of agents: (1) memantine and/or pharmacologically active metabolites, salts, solvates and racemates of memantine and (2) donepezil and/or pharmacologically active metabolites, salts, solvates and racemates of donepezil. In a specific preferred embodiment, the combination of agents comprises memantine and donepezil. In other embodiments, additional anti-Alzheimer's agents may be added to the combination.

The combination of agents may be administered using a variety of routes of administration known to those skilled in the art. Routes of administration include oral administration, preferably memantine in immediate release and donepezil in sustained release, preferably once or twice per day. In certain preferred embodiments, a pharmaceutical formulation comprising a combination of agents may be taken orally in the form of liquid, syrup, tablet, capsule, powder, sprinkle, chewtab, or dissolvable disk. Alternatively, pharmaceutical formulations of the present invention can be administered intravenously or transdermally. Additional routes of administration are known to those skilled in the art (see, e.g., Remington's Pharmaceutical Sciences, Gennaro A. R., Ed., 20.sup.th Edition, Mack Publishing Co., Easton, Pa.).

In some embodiments, the donepezil and memantine are formulated as a paste, jelly, or suspension. For example, the drugs are dissolved, entrapped or suspended in the form of drug particles, microencapsulated particles, or drug-polymer particles in a gelatinous solution or semi-solid. An advantage of an oral jelly formulation is that it is easier to administer the drugs to patients who have difficulty swallowing tablets, capsules or pills. In certain embodiments, both donepezil and mematine are thoroughly mixed and suspended in an appropriate medium to form a paste or a gel. Additional agents can optionally be mixed to provide flavor during oral administration. Peanut butter or alginate, flavored with raspberry and a sweetener are examples of the many suitable taste masking agents. In various embodiments, the paste or jelly can also be formulated with suitable binders or excipients known in the art for topical administration.

To measure the appropriate dose, the formulation is provided, for example, as a paste or jelly packaged in a tube similar to those used to dispense toothpaste or in a small cup. The paste can be squeezed out of an orifice of a fixed size such that a given length of paste contains a specified dose of donepezil and/or memantine, e.g., one inch contains 5 mg of donepezil, two inches contain 10 mg of donepezil, etc. An alternative approach is to package the jelly in a hypodermic syringe-like dispenser that is calibrated for dose. Yet another alternative approach is to package the formulation as a gelatinous solid, for example, a jelly candy bar. The bar can be scored or marked in units of donepezil and/or memantine for convenient dosing.

Methods of preparing sustained release formulations in the form of tablets, capsules or pills are known in the art. For example, U.S. Pat. Nos. 6,251,430 and 6,558,700 disclose a variety of methods that are herein incorporated by reference in their entirety. In some embodiments, the sustained release formulation is prepared by coating the active ingredient of the drug with a polymer, preferably a water-insoluble polymer. For example, a water-insoluble polymer used in the pharmaceutical field as a sustained release coating agent, an enteric coating agent, or a gastric coating agent. The water-insoluble polymer can include, for example, ethyl cellulose, purified shellac, white shellac, aminoalkyl methacrylate copolymer RS, hydroxypropyl methylcellulose phthalate, hydroxypropyl methylcellulose acetate succinate, carboxymethylethyl-cellulose, cellulose acetate phthalate, methacrylic acid copolymer L, methacrylic acid copolymer LD, methacrylic acid copolymer S, aminoalkyl methacrylate copolymer E, or polyvinyl acetal diethylaminoacetate.

The type, degree of substitution and molecular weight of the water-insoluble polymers can depend on solubility of the active ingredient in water or an alcohol, the desired sustained release level and the like. The water-insoluble polymers can be used either alone or in combination. There can be further incorporated a hydrogenated oil, stearic acid, or cetanol as a coating auxiliary agent, and a middle-chain triglyceride, triacetin, triethyl citrate, or cetanol as a plasticizer.

In some embodiments, the sustained release formulation is a matrix-type tablet or granule. The active ingredient can be coated with up to 3 different types of polymers. These three different types of polymers can include: 1) a water insoluble polymer, such as ethylcellulose; 2) a pH independent gelling polymer, such as hydroxypropyl methylcellulose; and 3) a pH dependent gelling polymer, such as sodium alginate. These three different types of polymers can be used together to attenuate the release rate of the drugs.

In a preferred embodiment, the donepezil 23 mg tablets are sustained release matrix-type tablets containing ethylcellulose and methacrylic acid copolymer type C, which provides a slow release of the active ingredient from a core matrix. In certain embodiments, three types of donepezil tablets are provided herein, designed to release the active ingredient as follows: over a 4-hour period (donepezil SR 4-hour); over an 8-hour period (donepezil SR 8-hour); and over a 12-hour period (donepezil SR 12-hour).

Dosage Forms: Release Properties

In a particular embodiment, provided herein is a method for delaying the symptomatic progression of a dementia associated with moderate to severe Alzheimer's disease, or treating such dementia comprising administering to a subject in need a combination therapy comprising an effective amount of memantine, and an effective amount of donepezil, wherein the memantine is in an immediate release form and the donepezil is in a sustained release form. Alternatively, both agents are formulated for sustained release.

A preferred pharmaceutical formulation includes approximately 20 mg of memantine formulated as an immediate release or sustained release formulation, and approximately 21-24 mg, e.g., 23 mg, of donepezil formulated as a sustained release formulation.

Sustained-release formulations can achieve a degree of sustained effect. However, the exposure and/or the bioavailability of the active ingredient may vary based on a variety of factors, such as for example, the absorption window, the carriers or excipients used in the formulation, the mode of delivery of the formulation, and/or the transit time of the active ingredient through the gastrointestinal tract of the patient.

In some embodiments, the sustained release formulation can include a higher amount, such as for example, 1-5 mg, to account for a loss in the bioavailability and to maintain an effective dose of the drug. For example, 24-28 mg of donepezil can be used to prepare a single formulation containing 23 mg of donepezil as a sustained release formulation and 10-20 mg of memantine as an immediate release formulation. In certain embodiments, up to 28 mg of memantine can be used to prepare a single formulation containing 23 mg of donepezil as a sustained release formulation and 20 mg of memantine as a sustained release formulation. In various embodiments, up to 32 mg of memantine can be used to prepare a single formulation containing 23 mg of donepezil as a sustained release formulation and 28 mg of memantine as a sustained release formulation.

The combination can be administered to provide a desired pharmacokinetic effect in a patient suffering from moderate to severe Alzheimer's, or dementia associated with moderate to severe Alzheimer's. Thus, in one aspect, provided herein is a method for treating moderate to severe Alzheimer's disease or dementia associated with moderate to severe Alzheimer's disease, comprising administering to a subject in need a combination therapy comprising an effective amount of memantine, and an effective amount of donepezil, wherein the donepezil is administered through a sustained release formulation capable of maintaining a substantially elevated serum level of the drug.

The combination therapy according to the present invention can contain at least one sustained-release portion for performing a sustained-release function and one immediate release portion for performing an immediate release function. In certain embodiments, when the combination therapy is in a single dosage form, it can be in the form of tablets formed from a mixture of sustained-release granules constituting a sustained-release portion and immediate-release granules constituting a immediate-release portion, a capsule preparation obtained by filling a capsule with sustained-release granules and immediate-release granules, or press-coated tablets in which an outer layer constituting a immediate-release portion is formed on an inner core constituting a sustained-release portion. There is, however, no limitation to the above embodiments. Moreover, there are no particular limitations on the state of containment of each anti-dementia drug in the composition or in a immediate-release portion or a sustained-release portion; the anti-dementia drug may be dispersed uniformly in the composition, immediate release portion or sustained release portion, or may be contained in only one part of the composition, immediate-release portion or sustained-release portion, or may be contained such that there is a concentration gradient.

A sustained-release portion in the composition according to the present invention can contain at least one non-pH-dependent polymeric substance or pH-dependent polymeric substance for controlling anti-dementia drug release.

The non-pH-dependent polymeric substance used in the present invention can comprise a polymeric substance whose charge state hardly changes under pH conditions generally found in the gastrointestinal tract, specifically from pH 1 to pH 8. This means, for example, a polymeric substance that does not have functional groups whose charge state changes depending on the pH such as basic functional groups such as amino groups or acidic functional groups such as carboxylic acid groups. Note that in the present invention, the non-pH-dependent polymeric substance can be included for giving the composition according to the present invention a sustained-release function, but may also be included for another purpose. Moreover, the non-pH-dependent polymeric substance used in the present invention may be water-insoluble, or may swell in water or dissolve in water to form a gel. Examples of water-insoluble non-pH-dependent polymeric substances include, but are not limited to, cellulose ethers, cellulose esters, and methacrylic acid-acrylic acid copolymers (trade name Eudragit, manufactured by Rohm GmbH & Co. KG, Darmstadt, Germany). Examples include, but are not limited to, cellulose alkyl ethers such as ethylcellulose (trade name Ethocel, manufactured by Dow Chemical Company, USA), ethyl methylcellulose, ethyl propylcellulose or isopropylcellulose, and butylcellulose, cellulose aralkyl ethers such as benzyl cellulose, cellulose cyanoalkyl ethers such as cyanoethylcellulose, cellulose organic acid esters such as cellulose acetate butyrate, cellulose acetate, cellulose propionate or cellulose butyrate, and cellulose acetate propionate, ethyl acrylate-methyl methacrylate copolymers (trade name Eudragit NE, manufactured by Rohm GmbH & Co. KG, Darmstadt, Germany), and aminoalkyl methacrylate copolymer RS (trade names Eudragit RL, Eudragit RS). There are no particular limitations on the mean particle diameter of a water-insoluble polymer used in the present invention, but usually the lower this mean particle diameter the better the performance, with the mean particle diameter preferably being from 0.1 to 100 μm, more preferably from 1 to 50 μm, particularly preferably from 3 to 15 μm, most preferably from 5 to 15 μm. Moreover, examples of water-soluble or water-swelling non-pH-dependent polymeric substances include, but are not limited to, polyethylene oxide (trade name Polyox, manufactured by Dow Chemical Company, molecular weight 100,000 to 7,000,000), low-substituted hydroxypropyl cellulose (trade name L-HPC, manufactured by Shin-Etsu Chemical, Japan), hydroxypropyl cellulose (trade name HPC, manufactured by Nippon Soda, Co., Ltd, Japan), hydroxypropyl methylcellulose (trade names Metolose 60SH, 65SH, 90SH, manufactured by Shin-Etsu Chemical, Japan), and methylcellulose (trade name Metolose SM, manufactured by Shin-Etsu Chemical, Japan).

Note that in the present invention, a single non-pH-dependent polymeric substance may be contained in the composition, or a plurality of the non-pH-dependent polymeric substances may be contained. The non-pH-dependent polymeric substance used in the present invention is preferably a water-insoluble polymeric substance, more preferably ethylcellulose, an ethyl acrylate-methyl methacrylate copolymer (trade name Eudragit NE), or an aminoalkyl methacrylate copolymer RS (trade name Eudragit RL, Eudragit RS). Particularly preferable is at least one of ethylcellulose and an aminoalkyl methacrylate copolymer RS. Most preferable is ethylcellulose. There are no particular limitations on the amount of the non-pH-dependent polymeric substance contained in the composition; this amount can be adjusted as appropriate in accordance with the purpose such as controlling sustained drug release.

A pH-dependent polymeric substance that can be used in the present invention is a polymeric substance whose charge state changes under pH conditions generally found in the gastrointestinal tract, specifically from pH 1 to pH 8. This means, for example, a polymeric substance having functional groups whose charge state changes depending on the pH such as basic functional groups such as amino groups or acidic functional groups such as carboxylic acid groups. The pH-dependent functional groups of the pH-dependent polymeric substance are preferably acidic functional groups, with the pH-dependent polymeric substance most preferably having carboxylic acid groups.

The pH-dependent polymeric substance used in the present invention may be water-insoluble, or may swell in water or dissolve in water to form a gel. Examples of pH-dependent polymeric substances used in the present invention include, but are not limited to, enteric polymeric substances. Examples of enteric polymeric substances include, but are not limited to, methacrylic acid-methyl methacrylate copolymers (Eudragit L100, Eudragit S100, manufactured by Rohm GmbH & Co. KG, Darmstadt, Germany), methacrylic acid-ethyl acrylate copolymers (Eudragit L100-55, Eudragit L30D-55, manufactured by Rohm GmbH & Co. KG, Darmstadt, Germany), hydroxypropyl methylcellulose phthalate (HP-55, HP-50, manufactured by Shin-Etsu Chemical, Japan), hydroxypropyl methylcellulose acetate succinate (AQOAT, manufactured by Shin-Etsu Chemical, Japan), carboxymethyl ethylcellulose (CMEC, manufactured by Freund Corporation, Japan), and cellulose acetate phthalate. Examples of pH-dependent polymeric substances that swell in water or dissolve in water to form a gel include, but are not limited to, alginic acid, pectin, carboxyvinyl polymer, and carboxymethyl cellulose. In the present invention, a single pH-dependent polymeric substance may be contained in the composition, or a plurality of pH-dependent polymeric substances may be contained. The pH-dependent polymeric substance used in the present invention is preferably an enteric polymeric substance, more preferably a methacrylic acid-ethyl acrylate copolymer, a methacrylic acid-methyl methacrylate copolymer, hydroxypropyl methylcellulose phthalate, or hydroxypropyl methylcellulose acetate succinate, particularly preferably a methacrylic acid-ethyl acrylate copolymer.

When using a pH-dependent polymeric substance in the manufacturing process of the composition according to the present invention, a commercially available product of a powder type or a granular type, or a suspension type in which the pH-dependent polymeric substance has been dispersed in a solvent in advance can be used as is, or such a commercially available product can be used dispersed in water or an organic solvent. The lower the particle diameter of the pH-dependent polymeric substance the better the performance, with the pH-dependent polymeric substance preferably being of the powder type. In the case of a methacrylic acid-ethyl acrylate copolymer, an example is Eudragit L100-55. There are no particular limitations on the mean particle diameter of a pH-dependent polymeric substance used in the present invention, but the mean particle diameter is preferably from 0.05 to 100 μm, more preferably from 0.05 to 70 μm, most preferably from 0.05 to 50 μm. Moreover, there are no particular limitations on the amount of the pH-dependent polymeric substance, for example, in the case of an enteric polymeric substance, the amount is generally from 0.1 to 90 parts by weight, preferably from 1 to 70 parts by weight, more preferably from 5 to 60 parts by weight, particularly preferably from 10 to 50 parts by weight, based on 100 parts by weight of the composition.

The combination therapy according to the present invention may further contain any of various additives, such as any of various pharmacologically acceptable carriers such as diluents, lubricants, binders and disintegrants, as well as preservatives, colorants, sweeteners, plasticizers, film coating agents and so on, as necessary. Examples of diluents include, but are not limited to, lactose, mannitol, dibasic calcium phosphate, starch, pregelatinized starch, crystalline cellulose, light silicic anhydride, synthetic aluminum silicate, magnesium aluminate metasilicate or the like. Examples of lubricants include, but are not limited to, magnesium stearate, calcium stearate, talc, sodium stearyl fumarate or the like. Examples of binders include, but are not limited to, hydroxypropyl cellulose, methylcellulose, sodium carboxymethyl cellulose, hydroxypropyl methylcellulose, polyvinylpyrrolidone or the like. Examples of disintegrants include, but are not limited to, carboxymethyl cellulose, calcium carboxymethyl cellulose, croscarmellose sodium, sodium carboxymethyl starch, low-substituted hydroxypropyl cellulose or the like. Examples of preservatives include, but are not limited to, paraoxybenzoic acid esters, chlorobutanol, benzyl alcohol, phenethyl alcohol, dehydroacetic acid, sorbic acid or the like. Preferable examples of colorants include, but are not limited to, water-insoluble lake pigments, natural pigments (e.g., β-carotene, chlorophyll, red ferric oxide), yellow ferric oxide, red ferric oxide, black ferric oxide or the like. Preferable examples of sweeteners include, but are not limited to, sodium saccharin, dipotassium glycyrrhizate, aspartame, stevia or the like. Examples of plasticizers include, but are not limited to, glycerol fatty acid esters, triethyl citrate, propylene glycol, polyethylene glycol or the like. Examples of film coating agents include, but are not limited to, hydroxypropyl methylcellulose, hydroxypropyl cellulose or the like.

Manufacturing Methods

To manufacture the combination therapy composition according to the present invention, a single conventional method, or a combination of conventional methods, can be used. For example, when manufacturing anti-dementia drug-containing granules as a sustained-release portion or an immediate-release portion in the present invention, granulation is the main operation, but this may be combined with other operations such as mixing, drying, sieving, and classification. As the granulation method, for example, a wet granulation method in which a binder and a solvent are added to the powder and granulation is carried out, a dry granulation method in which the powder is compressed and granulation is carried out, a molten granulation method in which a binder that melts on heating is added and heating and granulation are carried out, or the like can be used. Furthermore, in accordance with the granulation method, an operating method such as a mixing granulation method using a planetary mixer, a screw mixer or the like, a high-speed mixing granulation method using a Henschel mixer, a Super mixer or the like, an extruding granulation method using a cylindrical granulator, a rotary granulator, a screw extruding granulator, a pellet mill type granulator or the like, a wet high-shear granulation method, a fluidized-bed granulation method, a compression granulation method, a crushing granulation method, or a spraying granulation method can be used. After the granulation, drying using a dryer, a fluidized bed or the like, cracking, and sieving can be carried out to obtain the granules or fine granules for use. Moreover, a granulation solvent may be used when preparing the composition according to the present invention. There are no particular limitations on such a granulation solvent, which may be water or any of various organic solvents, for example, water, a lower alcohol such as methanol or ethanol, a ketone such as acetone or methyl ethyl ketone, methylene chloride, or a mixture thereof.

For sustained-release granules contained in the composition according to the present invention, at least one anti-dementia drug and at least one selected from non-pH-dependent polymeric substances and pH-dependent polymeric substances are mixed together, a diluent and a binder are added as necessary, and granulation is carried out to obtain granular matter. The granular matter obtained is dried using a tray dryer, a fluidized bed dryer or the like, and sieving is carried out using a mill or an oscillator, whereby the sustained-release granules can be obtained. Alternatively, as a method of manufacturing sustained-release granules in the present invention, it is possible to add at least one anti-dementia drug, at least one selected from non-pH-dependent polymeric substances and pH-dependent polymeric substances, and as necessary a diluent and a binder using a dry compactor such as a roller compactor or a slug tabletting machine, and carry out compression-molding while mixing, and then carry out granulation by cracking down to a suitable size. The granular matter prepared using such a granulator may be used as is as granules or fine granules according to the present invention, or may be further cracked using a power mill, a roll granulator, a rotor speed mill or the like, and sieved to obtain sustained-release granules. Note that immediate-release granules can also be manufactured as for the sustained-release granules.

A compression-molded product can be manufactured as an anti-dementia drug-containing sustained-release portion or immediate-release portion, or as the composition according to the present invention using a single conventional method, or a combination of conventional methods. For example, at least one anti-dementia drug, at least one selected from non-pH-dependent polymeric substances and pH-dependent polymeric substances, a diluent such as mannitol or lactose, a binder such as polyvinylpyrrolidone or crystalline cellulose, a disintegrant such as carmellose sodium or crospovidone, and a lubricant such as magnesium stearate or talc are used, and tableting is carried out using an ordinary method, whereby the compression-molded product can be obtained. In this case, tabletting is the main operation in the method of manufacturing the compression-molded product, but this may be combined with other operations such as mixing, drying, sugar coating formation, and coating. Examples of the method for the tabletting include, but are not limited to, direct compression molding in which at least one anti-dementia drug and pharmacologically acceptable additives are mixed together and then the mixture is directly compression-molded into tablets using a tabletting machine, and dry granule compression or wet granule compression in which sustained-release granules or immediate-release granules according to the present invention are subjected to compression-molding after adding a lubricant or a disintegrant as necessary. There are no particular limitations on the tabletting machine used in the compression molding; for example, a single-punch tabletting machine, a rotary tabletting machine, or a press-coated tabletting machine can be used.

The anti-dementia drug-containing sustained-release granules or immediate-release granules, or compression-molded product according to the present invention can be used as is in the form of granules or a tablet as the composition of the present invention, but may also be subjected to further processing to manufacture the composition. For example, the compression-molded product or granules can be given a film coating using a film base material such as ethylcellulose, casein, methylcellulose, hydroxypropyl methylcellulose, methacrylic acid copolymer L, cellulose acetate phthalate, shellac or the like, or given a sugar coating using a sugar coating liquid containing saccharose, sugar alcohol, gum arabic powder, talc or the like, thus producing film-coated tablets or sugar-coated tablets. A preferable solvent in this coating technique is purified water, but an organic solvent such as an alcohol, a ketone, an ether or a chlorinated hydrocarbon, or a mixture thereof can also be used. For example, ethanol, acetone, methylene chloride or the like can be used as an organic solvent. Moreover, as the coating apparatus, an apparatus ordinarily used in coating techniques for manufacturing medicines can be used, with examples including a spray coating apparatus in which the coating is carried out by spraying a coating liquid or the like, and a rotor fluidized bed granulator for layering.

In the case of manufacturing capsule preparations, the capsule preparations can be manufactured by filling sustained-release granules or immediate-release granules as above, or mini-tablets into hard gelatin capsules or HPMC capsules using an automatic capsule filling machine. Alternatively, in the case of the preparations for per-tube administration or a dry syrup that is used mixed with water or the like when taken, sustained-release granules or immediate-release granules as above can be mixed with a thickener or a dispersant so as to disperse these granules, the mixture then being made into granules or tablets. Furthermore, a liquid or jelly can be made using water, and substances selected from dispersants, emulsifiers, thickeners, preservatives, pH adjustors, sweeteners, flavorings, fragrances and so on. However, with respect to other manufacturing methods, there are no limitations to the above.

Dosage forms of both memantine and donepezil are further described in US Patent Publication no. 2009/0023778, which is incorporated herein by reference in its entirety.

The following examples illustrate the certain specific methods and compositions according to the present invention. A skilled artisan appreciates that the invention is not limited to the exemplary work described or to the specific details set forth in the examples.

### EXAMPLES

### Example 1

Donepezil SR 23 mg tablets are matrix-type tablets containing ethylcellulose and methacrylic acid copolymer, type C, which ensures a slow release of the active ingredient from the core matrix. Three types of donepezil SR tablets were initially formulated, designed to release the active ingredient as follows: over a 4-hour period (donepezil SR 4-hour); over an 8-hour period (donepezil SR 8-hour); and over a 12-hour period (donepezil SR 12-hour). Each of the three SR formulations contained varying amounts of the core matrix material ensuring different release profiles. In vitro, the rate of release of the active ingredient into the dissolution medium at pH 6.8 differed, while concentrations of the active ingredient achieved at the end of each release period were similar.

A bioavailability study (hereafter referred to as Study 020) in healthy subjects was conducted to compare the three dissolution types of donepezil SR as 10 mg tablets. Results of the study showed that donepezil SR 8-hour appears to be the optimal SR formulation compared to donepezil SR 4-hour and donepezil SR 12-hour, based on reductions in Cmax (>50%) and slightly reduced AUC, relative to equivalent doses of the IR formulation. Therefore, the SR 8-hour type was chosen for development. Subsequent Phase I studies were conducted using this formulation at doses of 14 mg and 23 mg.

A food-effect study (hereafter referred to as Study 023) was conducted in healthy subjects to evaluate the effect of food on the bioavailability of donepezil SR 23 mg following single and repeated oral administration. Results of this study showed consumption of a high fat meal prior to single-dose administration of donepezil SR 23 mg increased Cmaxvalues by approximately 22% but did not affect the AUC.

When subjects were administered donepezil SR under a modified multiple-dose regimen (donepezil SR 14 mg for 7 days, followed by a single dose of donepezil SR 23 mg) intended to reflect the conditions of the clinical use of the drug, no food effect was observed. Overall, food had no clinically significant effect on the rate or extent of drug absorption from the donepezil SR 23 mg formulation.

### Example 2

Approved therapies for moderate-to-severe Alzheimer's disease (AD) provide symptomatic benefit, without known clinical effects on disease progression. It is possible additional cholinesterase inhibition may mitigate the gradual loss of treatment benefit that occurs over time. To determine whether a higher (23 mg) once daily dose of donepezil could maintain or improve cognitive and global function, patients with moderate-to-severe AD (MMSE 0-20) treated ≧3 months with donepezil 10 mg were enrolled in a 24-week, global, double-blind, double-dummy, parallel-group clinical trial comparing once daily donepezil 23 mg extended-release tablets (23 mg) with continued treatment with once daily donepezil 10 mg immediate-release tablets (10 mg). The 23 mg extended-release formulation delays time to maximum plasma concentration and blunts Cₘₐₓ compared with 10 mg.

1467 patients were randomized 2:1, 23 mg:10 mg. Concomitant memantine was allowed. Co-primary endpoints were the Severe Impairment Battery (SIB, cognitive endpoint) and Clinician's Interview-Based Impression of Change plus caregiver input (CIBIC+, endpoint for global function). SIB was statistically significant favoring 23 mg among the intent-to-treat population (ITT) last observation carried forward (LOCF): LS mean change from baseline was 23 mg=2.6±0.58; 10 mg=0.4±0.66; difference=2.2; p=0.0001. Observed case (OC) results were similar. Scores at week 24 in the overall population for CIBIC+ were 23 mg=4.23±1.07; 10 mg=4.29±1.07 (p=0.1789). The difference in CIBIC+ approached statistical significance favoring 23 mg in the OC population (p=0.0592). Patients with more severe AD (MMSE 0-16) treated with 23 mg benefited significantly on both endpoints in the ITT-LOCF and OC populations: SIB p<0.0001; CIBIC+ p<0.03.

Overall, 23 mg administered once daily was well tolerated. Cholinergic-related AEs during the study were more common in with 23 mg, although no new safety signals emerged. AEs with a rate of ≧5% were nausea, vomiting, diarrhea, and anorexia. The discontinuation rate due to AEs in the 23 mg group (18.6%) was higher than in the 10 mg group (7.9%). The majority of these discontinuations in the 23 mg group occurred during initial exposure in the first month.

These data demonstrate that once daily donepezil 23 mg extended release can provide additional cognitive benefits to patients already on approved AD treatment. Patients who had more advanced AD appeared to benefit the most on measures of global function.

### Example 3

### Background

In vitro drug metabolism studies indicate that donepezil is metabolized primarily via CYP 3A4 and, to a lesser extent, via the polymorphic cytochrome P450 isozyme, CYP2D6.

Memantine is used to treat several neurological disorders, including Alzheimer's disease. In vitro studies conducted with marker substrates of CYP450 enzymes (CYP1A2, -2A6, -2C9, -2D6, -2E1, -3A4) showed minimal inhibition of these enzymes by memantine. In addition, in vitro studies indicate that at concentrations exceeding those associated with efficacy, memantine does not induce the cytochrome P450 isozymes CYP1A2, CYP2C9, CYP2E1 and CYP3A4/5. Therefore, interactions with drugs metabolized by these CYP enzymes, including donepezil, were not expected. Furthermore, memantine is predominantly eliminated via the renal route. Hence, drugs that are substrates and/or inhibitors of the CYP450 isozymes (such as donepezil) were not expected to alter the metabolism and disposition of memantine.

Objectives

The objectives of this supplementary population pK analysis are:

To use a previously developed model to evaluate the effect of co-administered memantine on the pK profile/exposure to donepezil in subjects with moderate to severe Alzheimer's disease (AD).

In case of a statistically significant effect, to examine whether the interaction further explains some of the inter-subject variability in donepezil oral clearance/exposure in AD patients following the co-administration of memantine.

Methods

The final database used for model building and evaluation consisted of 3445 concentrations from a total of 850 subjects, of whom 308 were males and 542 females with an average age and weight of 74.3±8.29 y and 66.8±14.3 kg, respectively. There were 560 patients who were not taking concomitant memantine and 290 patients who were taking memantine. The patients taking memantine were administered a 10 mg dose of memantine, twice daily, as an immediate release formulation.

There was an average of 4.1 observations per subject in this sparse Phase III database. Data from the Phase III study were then fitted using the same structural model determined in the preliminary Phase I evaluation using NONMEM version VI Level 2 (ICON, Hanover Md., USA). Model building and covariate assessments were conducted using standard methods. The final model for the Phase III database was evaluated for performance using several tests, including evaluation of an internal validation database, and nonparametric bootstrap.

Updated Donepezil Population Pharmacokinetic Model

The updated best final pharmacokinetic model for donepezil was a two compartment model with transit input and linear elimination. The model was parameterized for the apparent clearance (CL/F), the apparent volumes of distribution of the central (Vc/F) and peripheral (Vp/F) compartments and the transit rate constant for the IR formulation (Ktr) and the SR formulation (KtrSR). The model included variance terms for CL/F, Vc/F, Vp/F and Ktr. The base model was developed for the Phase 1 database and was used as a frequentist prior to stabilize the subsequent model developed for Phase III data.

The final population PK model for donepezil included the effects of gender, weight, age, CYP2D6 genotype status, administration of CYP2D6 inhibitors, co-administration of memantine on CL/F and body weight on Vc/F. The bioavailability (F) of the SR relative to the IR formulation was also estimated. Donepezil CL/F was statistically significantly 21% lower in subjects receiving donepezil concomitantly with memantine.

The parameter estimates for the final model are presented in the table below. There was little difference between the parameter estimates reported originally and the updated parameter estimates. The parameters were estimated with good precision, and the standard errors (SE) were generally low (<19%). For a typical subject, the model parameters would predict a terminal elimination half-life of approximately 71.5 hours, which is consistent with reported values. When memantine is co-administered, the terminal elimination half life of donepezil increases to approximately 89 hours.

TABLE 1
Parameter Estimates for Updated Final Donepezil
Pharmacokinetic Model-Phase III Database

| | | | | % CV Inter- | |
|---|---|---|---|---|---|
| Parameter | | Population | | Individual | |
| (Units) | | Mean (SE*) | | Variance (SE*) | |
| CL/F_{2D6,EM} (L/h) | Θ₁ | 10.5 | (2.2) | 31.6 | (5.2) |
| CL/F_{2D6,PM} (L/h) | Θ₈ | 7.14 | (6.7) | | |
| CL/F_{2D6,URM} (L/h) | Θ₉ | 13.6 | (10.4) | | |
| Effect of Weight | — | 0.75 | FIXED | | |
| Effect of Gender | Θ₁₀ | 0.877 | (2.4) | | |
| Effect of Age | Θ₁₁ | −0.545 | (18.9) | | |
| Effect of CYP2D6 Inhibitor | Θ₁₃ | 0.868 | (3.8) | | |
| Effect of memantine | Θ₁₄ | 0.211 | (9.5) | | |
| Vc/F (L) | Θ₂ | 539 | (3.5) | 47.6 | (8.4) |
| Effect of Weight | — | 1 | FIXED | | |
| Q/F (L/h) | Θ₃ | 35.9 | (1.7) | NE | |
| Vp/F (L) | Θ₄ | 625 | (0.7) | 646 | (18.7) |
| Ktr (1/h) | Θ₅ | 2.76 | (2.2) | 33.9 | (8.3) |

| | | | | |
|---|---|---|---|---|
| KtrSR (1/h) | Θ₆ | 0.888 | (2.2) | |
| Relative Bioavailability SR | Θ₁₂ | 0.912 | (1.4) | NE |

| | |
|---|---|
| CCV Residual Error (as % CV) | 29.6 (1.5) |

*SE given as % CV (parameter SE/parameter estimate);
NE—Not Estimated

Cₘₐₓ values refer to the maximum observed plasma concentration of the active drug, for example, donepezil hydrochloride. Tₘₐₓ values refer to the time of maximum observed plasma concentration. The plasma concentrations of donepezil increased with increasing dose over the course of the study for subjects that were administered active treatment throughout the study. Based on historical (steady-state) data for the immediate release (IR) formulation, concentrations of donepezil following donepezil sustained release (SR) 23 mg tablet administration approached steady-state values during 14 days of repeated dose administration, similar to donepezil IR. After 14 days of daily administration with donepezil SR 23 mg, the Tₘₐₓ was 5.2 hours which is longer than the historical IR formulation data (approximately 3 hours). Cₘₐₓ values obtained after repeated-dosing with donepezil SR 23 mg tablets were higher than after administration of a single-dose while Tₘₐₓ values were similar. The Cₘₐₓ values ranged from about 32.63 ng/mL for a single dose administration to about 129.2 ng/mL for repeated administration. The corresponding Tₘₐₓ values ranged from about 6.1 h for a single dose administration to about 5.2 h for repeated administration.

FIG. 3 compares increases in dose normalized donepezil concentration between patients receiving a once daily dose of 23 mg of donepezil (sustained release) along with a twice daily dose of 10 mg each of memantine (immediate release) vs. patients receiving only the once daily dose of 23 mg of donepezil (sustained release). As can be seen, the concentration of donepezil in the blood plasma were higher at all times of day at steady state in patients who were also taking memantine indicating a slower metabolism of donepezil when administered in combination with memantine. A log-linear plot of the dose normalized concentration versus time for the SR and IR formulations stratified on whether the patient was or was not taking memantine is shown. In this figure, the mean dose-normalized donepezil concentrations are higher when donepezil is co-administered with memantine than when it is administered as a single agent. This finding is consistent with the covariate factor identified in the model-based evaluation.

FIG. 4 compares increase in dose normalized donepezil concentration between patients receiving a once daily dose of 10 mg of donepezil (immediate release) along with a twice daily dose of 10 mg each of memantine (immediate release) vs. patients receiving only the once daily dose of 10 mg of donepezil (immediate release). Again, the concentration of donepezil in the blood plasma were higher at all times of day at steady state in patients who were also taking memantine indicating a slower metabolism of donepezil when administered in combination with memantine. A sustained release formulation, once daily, containing 23 mg of donepezil hydrochloride was administered to patients. The dotted line indicates patients who did not also receive any memantine hydrochloride. The bold line indicates patients who also received an immediate release formulation, twice daily, containing 10 mg of memantine hydrochloride, i.e., a total of 20 mg memantine hydrochloride per day.

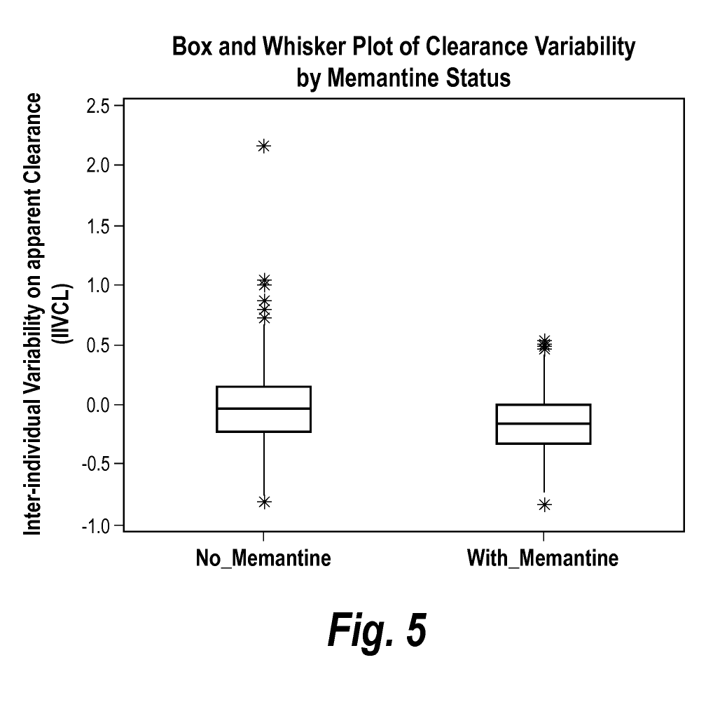

FIG. 5 shows a box and whisker plot of the inter-individual variability on apparent clearance for both immediate release and sustained release formulations versus memantine co-administration status. The central line in the box represents the median value in the distribution of individual values, the lower edge of the box represents the 25^{th} percentile, the upper edge of the box represents the upper 75^{th} percentile and the lower and upper “whiskers” or lines radiating from the box represent the lower 2.5^{th} percentile and the upper 97.5^{th} percentile, respectively. The stars represent outliers in each distribution as determined by the statistical program used to generate the figure. As can be seen, the median value of donepezil's apparent clearance for patients taking memantine is lower than for patients not taking memantine. The median value of donepezil apparent clearance for patients taking memantine is lower than for patients not taking memantine. This additional evaluation of the population pharmacokinetics of donepezil in patients diagnosed with Alzheimer's Disease revealed a 21% lower apparent clearance in patients receiving donepezil with concomitant memantine compared to patients administered donepezil alone (95% CI: 13.8-24.2%), an unexpected effect that would result in a higher donepezil exposure.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein can be utilized according to such related embodiments of the present invention. Thus, the following claims are intended to encompass within their scope modifications, substitutions, and variations to processes, manufactures, compositions of matter, compounds, means, methods, and/or steps disclosed herein.

1. A method of treating moderate to severe Alzheimer's disease in a patient in need thereof, comprising administering to the patient a daily dose of (i) 21-24 mg of donepezil, or any pharmaceutically acceptable salt thereof, and (ii) 20 mg of memantine, or any pharmaceutically acceptable salt thereof.

2. The method of claim 1, comprising administering 23 mg of donepezil.

3. The method of claim 1, wherein the donepezil is formulated as an immediate release formulation.

4. The method of claim 1, wherein the donepezil is formulated as a sustained release formulation.

5. The method of claim 1, wherein the donepezil is formulated as an immediate release formulation comprising 23 mg of donepezil.

6. The method of claim 1, wherein the donepezil is formulated as a sustained release formulation comprising 23 mg of donepezil.

7. The method of claim 1, wherein the patient has a Mini-Mental State Exam (MMSE) score of 0-20.

8. The method of claim 1, wherein the patient has a Mini-Mental State Exam (MMSE) score of 0-16.

9. The method of claim 1, wherein the patient has a Mini-Mental State Exam (MMSE) score of 0-10.

10. The method of claim 1, wherein the memantine is formulated as an immediate release formulation comprising 10 mg of memantine administered twice daily.

11. The method of claim 1, wherein the memantine is formulated as a sustained release formulation comprising 10 mg of memantine administered twice daily.

12. The method of claim 1, wherein the memantine is formulated as a sustained release formulation comprising 20 mg of memantine.

13. The method of claim 1, wherein the donepezil and memantine are administered simultaneously, separately or sequentially.

14. The method of claim 1, wherein the daily dose of memantine comprises two unit doses of 10 mg each.

15. The method of claim 1, wherein the donepezil and memantine are formulated as a single formulation.

16. The method of claim 1, wherein the donepezil is donepezil hydrochloride.

17. The method of claim 1, wherein the memantine is memantine hydrochloride.

18. The method of claim 1, wherein the patient has severe Alzheimer's disease.

19. The method of claim 1, wherein the treatment comprises slowing the symptomatic progression of the moderate to severe Alzheimer's disease.

20. A method of treating moderate to severe Alzheimer's disease in a patient with a Mini-Mental State Exam (MMSE) score of 0-16, comprising administering to the patient (i) a sustained release formulation comprising 23 mg of donepezil, or any pharmaceutically acceptable salts thereof, once daily and (ii) an immediate release formulation comprising 10 mg of memantine, or any pharmaceutically acceptable salts thereof, twice daily.

21. A method of treating moderate to severe Alzheimer's disease in a patient with a Mini-Mental State Exam (MMSE) score of 0-16, comprising administering to the patient a sustained release formulation comprising (i) 23 mg of donepezil, or any pharmaceutically acceptable salts thereof, and (ii) 20 mg of memantine, or any pharmaceutically acceptable salts thereof, wherein the formulation is a single formulation or unit dose.

22. A pharmaceutical formulation comprising (i) 21-24 mg of donepezil, or any pharmaceutically acceptable salts thereof, (ii) 20 mg of memantine, or any pharmaceutically acceptable salts thereof, and (iii) a pharmaceutically acceptable carrier or excipient, wherein the formulation is a single formulation or unit dose.

23. The pharmaceutical formulation of claim 22, wherein the formulation is an immediate release formulation.

24. The pharmaceutical formulation of claim 22, wherein the donepezil is formulated as a sustained release formulation and the memantine is formulated as an immediate release formulation.

25. The pharmaceutical formulation of claim 22, wherein the formulation is a sustained release formulation.

26. The pharmaceutical formulation of claim 22, wherein the donepezil is donepezil hydrochloride.

27. The pharmaceutical formulation of claim 22, wherein the memantine is memantine hydrochloride.

28. A sustained release formulation comprising (i) 23 mg of donepezil hydrochloride, (ii) 28 mg of memantine hydrochloride, and (iii) a pharmaceutically acceptable carrier or excipient, wherein the formulation is a single formulation or unit dose.
